(19) United States
(12) Patent Application Publication
(10) Pub. No.: US 2013/0164274 A1
(43) Pub. Date: Jun. 27, 2013

(54) KIT FOR MONITORING, DETECTING AND STAGING GVHD

(71) Applicant: Stephen G. Marx, Jerusalem (IL)

(72) Inventor: Stephen G. Marx, Jerusalem (IL)

(21) Appl. No.: 13/821,311

(22) PCT Filed: Sep. 7, 2011
(86) PCT No.: PCT/IL11/00711

§ 371 Date: Mar. 7, 2013

Related U.S. Application Data
(60) Provisional application No. 61/380,337, filed on Sep. 7, 2010.
(60) Provisional application No. 61/433,540, filed on Jan. 18, 2011.

(51) Int. Cl. G01N 33/68

(52) U.S. Cl. CPC G01N 33/6893
USPC 424/94.5; 506/18; 506/9; 436/501

(57) ABSTRACT

A method and kit for detecting Graft versus Host Disease (GVHD) in a mammal including a human comprising the steps of; obtaining a sample from the patient or subject; measuring the level of GSK-3.beta. inactivation by measuring beta-catenin levels in the absence of serine 9 phosphorylation, or by measuring the site of stimulation on the GSK-3beta once stimulated through the activated Wnt pathway, or by measuring a protein marker that is unique to the inactivation of GSK-3 beta through the activated Wnt pathway, comparing the result of said inactivation levels with a predetermined normal inactivation value obtained from healthy subjects, such that a deviation of at least about 25% from normal inactivation values indicates the presence of GVHD.

### BACKGROUND OF THE INVENTION

1. Field of the Invention

The present invention is in the field of diagnostic and regulatory kits for the treatment of conditions associated with pathological cell proliferation. More specifically, the invention is adapted to measure and regulate the activation and inactivation levels of glycogen synthase kinase 3 (GSK-3) as a marker of T-cell proliferation, especially in conditions such as Graft versus Host Disease (GVHD).

2. Background Art

Glycogen synthase kinase 3 (GSK-3) is a serine/threonine kinase that is known to act as a downstream regulatory switch that determines the output of numerous signaling pathways initiated by diverse stimuli. Through these pathways GSK-3 plays a central role in cellular proliferation and apoptosis. The ability to detect the activation state of the GSK-3 and if necessary to regulate it, would allow a greater understanding and ability to treat and diagnose conditions related to cell proliferation and apoptosis.

In many conditions related to the immune response, including tissue rejection, inflammation, infection and GVHD, there is a marked change in cell proliferation, in particular an increase in T-cell proliferation.

The incidence of GVHD has increased substantially over recent years. The number of allogeneic hematopoietic stem cell transplants (HSCT) that occur annually continues to increase each year. GVHD may also occur occasionally as a complication of blood transfusion. Clinically, the diagnosis of GVHD is suspected when the patient develops one or more of the following observable symptoms: dermatitis (skin rash), cutaneous blisters, crampy abdominal pain with or without diarrhea, persistent nausea and vomiting, hepatitis (with elevation of bilirubin and/or liver enzymes) (Jacobsohn D A and Vogelsang G B 2007). Clinical observations along with current diagnostic techniques are time-consuming and require invasive tissue sampling, involving tissue biopsies obtained from an involved organ in order to confirm and diagnose GVHD. Although biopsies are the only way to properly diagnose GVDH today, the diagnosis is determined only after T cells are already attacking the host tissues and the GVHD process has reached phase 3 of the disorder. Biopsy procedures to procure tissue are also invasive and incur delays of 24 to 48 hours before a diagnosis can be rendered. Furthermore, an unequivocal diagnosis is not always possible with biopsies, and diagnosis from clinical symptoms is not reliable, given that other post-transplant conditions may present in a similar manner.

There is therefore a long-felt need to provide a rapid, reliable relatively non-invasive method for detecting GVHD and other pathological conditions involving cell proliferation in the early stages of the disease process.

### SUMMARY OF THE INVENTION

The present invention relates to the field of diagnostic and regulatory kits and more specifically to a diagnostic and regulatory kit for Graft versus Host Disease.

It is one object of the present invention to disclose a method of detecting GVHD in a mammal including a human comprising the steps of obtaining a sample from the patient or subject; and, measuring the level of GSK-3β; wherein a significant deviation from normal values indicates the presence of GVHD.

It is yet another object of the present invention to disclose the method as defined above, further comprising steps of measuring GSK-3β expression levels.

It is yet another object of the present invention to disclose the method as defined above, further comprising steps of measuring GSK-3β activation levels.

It is yet another object of the present invention to disclose the method as defined above, further comprising steps of measuring GSK-3β inactivation levels.

It is yet another object of the present invention to disclose the method as defined above, further comprising steps of measuring serine 9 phosphorylated GSK-3β expression.

It is yet another object of the present invention to disclose the method as defined above, further comprising steps of measuring p-PKC expression.

It is yet another object of the present invention to disclose the method as defined above, further comprising steps of sampling the spleen.

It is yet another object of the present invention to disclose the method as defined above, further comprising steps of sampling the blood.

It is yet another object of the present invention to disclose the method as defined above, further comprising steps of sampling a body fluid or tissue.

It is yet another object of the present invention to disclose the method as defined above, further comprising steps of comparing the result of said GSK-3β levels with a predetermined normal value obtained from healthy subjects.

It is yet another object of the present invention to disclose the method as defined above, further comprising steps of comparing the result of said GSK-3β levels with a predetermined normal value obtained from healthy subjects, wherein a deviation of at least about 25% from normal inactivation values indicates the presence of GVHD.

It is yet another object of the present invention to disclose the method as defined above, comprising the steps of; obtaining a sample from the patient or subject, measuring the level of GSK-3β, comparing the result of said levels with a predetermined normal value obtained from healthy subjects, such that a deviation of at least about 25% from normal values indicates the presence of GVHD.

It is yet another object of the present invention to disclose a method of detecting GVHD in a mammal including a human comprising the steps of; obtaining a sample from the patient or subject, measuring the level of GSK-3β inactivation by measuring β-catenin levels in the absence of serine 9 phosphorylation, or by measuring the site of stimulation on the GSK-30 once stimulated through the activated Wnt pathway, or by measuring a protein marker that is unique to the inactivation of GSK-3β through the activated Wnt pathway, comparing the result of said inactivation levels with a predetermined normal inactivation value obtained from healthy subjects, such that a deviation of at least about 25% from normal inactivation values indicates the presence of GVHD.

It is yet another object of the present invention to disclose a method of detecting GVHD in a mammal including a human comprising the steps of obtaining a sample from the patient or subject; measuring the level of GSK-3β inactivation by measuring a protein marker that is unique to the inactivation of GSK-3β through the activated PKC; comparing the result of said inactivation levels with a predetermined normal inactivation value obtained from healthy subjects, wherein a significant deviation from normal inactivation values indicates the presence of GVHD.

It is yet another object of the present invention to disclose a method of detecting GVHD as defined above, further comprising steps of sampling the blood.

It is yet another object of the present invention to disclose a method of detecting GVHD as defined above, further comprising steps of sampling the spleen.

It is one object of the present invention to disclose a diagnostic kit for the identification and diagnosis of a pathological process associated with GVHD, in which the diagnostic molecule is GSK-3β.

It is yet another object of the present invention to disclose a diagnostic kit for the identification and diagnosis of a pathological process associated with GVHD wherein the kit is adapted for detecting the expression level of GSK-3β.

It is yet another object of the present invention to disclose a diagnostic kit for the identification and diagnosis of a pathological process associated with GVHD wherein the kit is adapted to detecting expression of p-PKC.

It is yet another object of the present invention to disclose a diagnostic kit for the identification and diagnosis of a pathological process associated with GVHD, such that the activation status of GSK-3β is a determinant of said pathological process.

It is also in the scope of the present invention to disclose a diagnostic kit for the detection of GVHD such that GVHD is detectable during phase 2 of the disease.

It is also in the scope of the present invention to disclose a diagnostic kit for the detection of GVHD such that the activation and/or expression status of GSK-3β is a marker for the progression and/or presence of GVHD.

It is also in the scope of the present invention to disclose a diagnostic kit in which the pathological condition is tissue rejection.

In yet another preferred embodiment of the present invention, a diagnostic kit for the detection of GVHD is disclosed, in which the activation and/or expression status of GSK-3β is a marker for the progression and/or presence of GVHD.

In yet another preferred embodiment of the present invention, a diagnostic kit for the detection of GVHD is disclosed, in which the activation status and/or expression level of GSK-3β is determined by examining markers for the Wnt pathway or the actual site on the GSK-3β being affected by the activated Wnt pathway.

It is yet another object of the present invention to disclose a diagnostic kit for the detection of GVHD, in which the activation status and/or expression level and/or expression level of GSK-3β is a marker for the progression and/or presence of GVHD, further wherein the activation status of GSK-3β is determined by examining serine 9 phosphorylation markers on the GSK-3β molecule.

In another aspect of the present invention, a diagnostic kit for the detection of GVHD is disclosed, in which the activation status and/or expression level of GSK-3β is a marker for the progression or presence of GVHD, and in which furthermore the activation status and/or expression level of GSK-3β is determined by measuring PKC activation sites on GSK-3β, PKC expression and/or a protein marker specific for activated PKC inactivation of the GSK-3β.

It is also in the scope of the present invention to disclose a diagnostic kit for the detection of GVHD, in which over-activation of GSK-3β is determined by measuring tyrosine 216 phosphorylation.

It is also in the scope of the present invention to disclose a diagnostic kit for the detection of GVHD, in which over-activation of GSK-3β is determined by measuring the expression levels of GSK-3β.

In yet another aspect of the present invention, a diagnostic kit for the detection of GVHD is disclosed, in which the presence of over-inactivated GSK-3β as indicated by abnormal levels of phosphorylated serine 9 provides a positive diagnosis of GVHD.

In yet another preferred embodiment of the present invention, a diagnostic kit for the detection of GVHD is disclosed, provided with the means for detecting the presence of over-inactivated GSK-3β as indicated by abnormal levels of β-catenin in the absence of serine 9 phosphorylation, or as indicated by abnormal levels of stimulation at the site on the GSK-3β affected by the activated Wnt pathway, or as indicated by abnormal levels of a protein marker that is unique to the inactivation of GSK-3β through the activated Wnt pathway thereby providing a positive diagnosis of GVHD.

It is yet another object of the present invention to disclose a diagnostic kit for the detection of GVHD, provided with the means for detecting the presence of over-inactivated GSK-3β as indicated by abnormal levels of stimulation at the site on the GSK-3β affected by activated PKC, or as indicated by abnormal levels of a protein marker that is unique to the inactivation of GSK-3β through activated PKC thereby providing a positive diagnosis of GVHD.

It is yet another object of the present invention to disclose a diagnostic kit for the detection of GVHD, provided with the means for detecting the presence of over-inactivated GSK-3β as indicated by abnormal levels of phosphorylated serine 9; and by abnormal levels of markers for Wnt activity including but not limited to abnormal levels of β-catenin expression, abnormal levels of stimulation at the site on the GSK-3β affected by the activated Wnt pathway, abnormal levels of protein markers unique to the inactivation of GSK-3β through the activated Wnt pathway; or any combination thereof, thereby providing a positive diagnosis of GVHD.

It is yet another object of the present invention to disclose a diagnostic kit for the detection of GVHD, provided with the means for detecting the presence of over-inactivated GSK-3β as indicated by abnormal levels of phosphorylated serine 9; and by abnormal levels of markers for activated PKC inactivation of the GSK-3β including but not limited to abnormal levels of stimulation at the site on the GSK-3β affected by the activated PKC, and abnormal levels of protein markers unique to the inactivation of GSK-3β through activated PKC, or any combination thereof, thereby providing a positive diagnosis of GVHD.

In yet another preferred embodiment of the present invention a diagnostic kit for the detection of GVHD is disclosed, provided with the means for detecting the presence of over-inactivated GSK-3β as indicated by abnormal levels of markers for Wnt activity including but not limited to abnormal levels of β-catenin expression, abnormal levels of stimulation at the site on the GSK-3β affected by the activated Wnt pathway and abnormal levels of protein markers unique to the inactivation of GSK-3β through the activated Wnt pathway; and by abnormal levels of markers for activated PKC inactivation of the GSK-3β including but not limited to abnormal levels of stimulation at the site on the GSK-3β affected by the activated PKC, and abnormal levels of protein markers unique to the inactivation of GSK-3β through activated PKC; or any combination thereof, thereby providing a positive diagnosis of GVHD.

In yet another aspect of the present invention a diagnostic kit for the detection of GVHD is disclosed, provided with the means for detecting the presence of over-inactivated GSK-3β as indicated by abnormal levels of phosphorylated serine 9 and abnormal levels of markers for Wnt activity including but not limited to abnormal levels of β-catenin expression, abnormal levels of stimulation at the site on the GSK-3β affected by the activated Wnt pathway, and abnormal levels of protein markers unique to the inactivation of GSK-3β through the activated Wnt pathway; and by abnormal levels of markers for activated PKC inactivation of the GSK-3β including but not limited to abnormal levels of stimulation at the site on the GSK-3β affected by the activated PKC, and abnormal levels of protein markers unique to the inactivation of GSK-3β through activated PKC; or any combination thereof, thereby providing a positive diagnosis of GVHD.

It is yet another object of the present invention to disclose a diagnostic kit for GVHD, in which the diagnostic kit characterizes the activation status of GSK-3β so as to determine the cause of the GVHD.

It is yet another object of the present invention to disclose a diagnostic kit for GVHD, in which the diagnostic kit characterizes the expression status of GSK-3β so as to determine the cause of the GVHD.

In yet another preferred embodiment of the present invention, a method of diagnosing GVHD by determining expression and/or inactivation state of GSK-3β is disclosed, by measuring at least one inactivation indicator selected from the group including serine 9 phosphorylation, Wnt activation as determined by β-catenin levels or through a protein marker specific for inactivation of the GSK-3β through the activated Wnt pathway or through the site on the GSK-3β that the activated Wnt pathway affects to stimulate inactivation of the GSK-3β; PKC expression levels, PKC inactivation sites on GSK-3β or through a protein marker specific for inactivation of the GSK-3β through activated PKC, or any combination thereof.

It is another object of the present invention to disclose a method of diagnosing GVHD by measuring expression and/or activation of GSK-3β by measuring at least one activation indicator selected from the group including over-expression of GSK-3β, tyrosine 216 phosphorylation or any combination thereof.

In another preferred embodiment of the present invention, a method of diagnosing GVHD in a mammal including a human is disclosed comprising the steps of; measuring GSK-3β inactivation indicators, measuring GSK-3β activation indicators, combining measurements of activation and inactivation indicators, such that activation to inactivation ratio is obtained characteristic of GVHD.

In yet another aspect of the present invention a diagnostic kit for determining the presence of GVHD by monitoring the activation status and/or expression level of GSK-3β is disclosed in which the diagnostic kit uses further protein markers to further distinguishes between GVHD and other pathological or normal physiological conditions that are also characterized by increased cell proliferation, decreased cell apoptosis and an immune response.

It is another object of the present invention to disclose a diagnostic kit for determining the presence of GVHD, in which an additional positive detection of a unique RNA and/or protein profile for GVHD further verifies the presence of said GVHD during phase 2 of the disease.

In yet another preferred embodiment of the present invention, a diagnostic kit for GVHD is disclosed in which the results provided by said kit indicate the pathways to be modulated to control the activation status of GSK-3β, thereby regulating GVHD.

It is yet another object of the present invention to disclose a diagnostic kit for the detection of the activation status and/or expression levels of GSK-3β, in which the results of the activation status and/or expression level measurement are an indicator for the progression/presence of GVHD.

It is yet another object of the present invention to disclose a diagnostic kit for the detection and quantification of the activation status and/or expression levels of GSK-3β, in which said activation status is an indicator of the cause of GVHD and furthermore in which said activation status and/or expression level is an indicator for the best treatment program.

It is yet another object of the present invention to disclose a diagnostic kit for the detection of GVHD, in which the GVHD is diagnosed by determining the activation status and/or expression levels of GSK-3β in blood samples from a mammal including a human patient.

It is yet another object of the present invention to disclose a protein chip comprising an array of bound antibodies, biomarkers or antigens useful for determining the expression levels of different GVHD relevant proteins in a given sample.

It is yet another object of the present invention to disclose a protein chip as defined above, comprising antibodies, biomarkers or antigens characteristic of the activation state and/or expression level of GSK-3β, related pathways, apoptosis markers, proliferation markers, and other biological and biochemical molecules relevant to GVHD wherein said antibodies, biomarkers or antigens are detectable before appearance of GVHD symptoms in a mammal including a human.

It is yet another object of the present invention to disclose a protein chip as defined above, wherein said antibodies, biomarkers or antigens are selected from the group comprising Total GSK-3β, ser 9 phosphorylated GSK-3β, GSK-3β ser 9 phosphorylation PKC Activation, b-catenin, Dvl, Akt, Erk, P38 MAPK, GSK-3β Tyrosine 216 Phosphorylation, Serum Albumin, markers for apoptosis comprising Fas, Bcl family, Cyctochrome C and caspases, markers for immune activity comprising Nf-kB and CD25, markers for proliferation comprising Cyclin D1, PCNA and p27), and any combination thereof.

In is yet another preferred embodiment of the present invention a method of regulating the activation status and/or expression level of the GSK-3β in a mammal including a human is disclosed comprising the steps of; obtaining a blood sample from a patient or subject, determining the activation status and/or expression level of GSK-3β, regulating the activation status and/or expression level of GSK-3β according to the findings of the previous step, and obtaining a normal activation status and/or expression level for GSK-3β such that regulation of said activation status and/or expression level of the GSK-3β is adapted for controlling GVHD.

It is one object of the present invention to disclose a regulatory and therapeutic kit useful for controlling GVHD, in which the therapeutic molecule is GSK-3β.

### BRIEF DESCRIPTION OF THE DRAWINGS

The invention is herein described, by way of example only, with reference to the accompanying drawings. The particulars shown are by way of example only and for the purposes of illustrative discussion of the preferred embodiments of the present invention only. The description taken with the drawings should make apparent to those skilled in the art how several forms of the invention may be embodied in practice.

In the drawings:

FIG. 1 is a pictorial illustration of the GSK3α and GSK3β subunits and their known activation and inactivation sites;

FIG. 2 is a flow diagram connecting GSK3β to GVHD;

FIG. 3 is a pictorial illustration of the disease process in GVHD;

FIG. 4A is a graphical representation of ser 9-phosphorylated GSK-3β (p-GSK-3β) protein expression levels in spleen samples;

FIG. 4B is a graphical representation of total GSK-3β protein expression levels in spleen samples;

FIG. 5 is a graphical representation of phosphorylated PKC (p-PKC) protein expression levels in spleen samples;

FIG. 6A is a graphical representation of ser 9-phosphorylated GSK-3β (p-GSK-3β) protein expression levels in blood samples; and,

FIG. 6B is a graphical representation of total GSK-3β protein expression levels in blood samples.

### DETAILED DESCRIPTION OF THE INVENTION

The present invention solves many of the problems of current methods and systems for monitoring patients suffering from conditions associated with pathological cell proliferation and pathological cell apoptosis especially GVHD.

It is therefore a preferred embodiment of the present invention to disclose a diagnostic kit, capable of detecting the expression level of glycogen synthase kinase 3β (GSK-3β. In this embodiment the GSK-3β expression level is an indicator of GVHD in the patient.

GSK-3β is now known to act as a downstream regulatory switch that determines the output of numerous signaling pathways initiated by diverse stimuli (Frame and Cohen 2001, Grimes and Jope 2001, Woodgett 2001 and Doble and Woodgett 2003). The activation status of the GSK-3β, the expression levels of the GSK-3β or the site on the GSK-3β responsible for activating or inactivating of the GSK-3β, are used to detect and monitor GVHD.

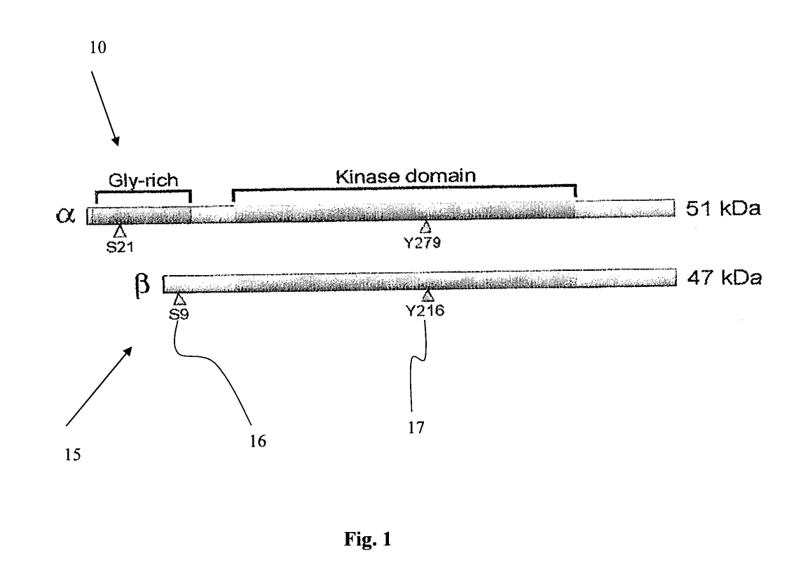

Reference is now made to FIG. 1 in the drawings, which shows the two subunits of GSK-3, GSK-3α 10 and GSK-3β 15 and their phosphorylation sites. Specific reference is made to the GSK-3β subunit, in which known phosphorylation sites, including the serine 9 (S9) 16 and tyrosine 216 (Y216) 17, are shown. In this embodiment the normal physiological state of the GSK-3β is in an unphosphorylated or dephosphorylated active state. In this embodiment serine 9 phosphorylation inactivates the GSK-3β, which can be identified and quantitated according to the methods described forthwith in the description. Serine 9 phosphorylation is associated with various other proteins including but not limited to cyclic adenosine monophosphate (cAMP), Phosphoinositide 3-kinase (PI3K), Phosphatidylinositol 4,5-biphosphate (PIP₂), protein kinase A (PKA) and Akt (serine/threonine protein kinase family).

In a further embodiment of the present invention, GSK-3β inactivation may also occur through the Wnt signaling pathway. Wnt activation inactivates the GSK-3β through an inactivation site independent of serine 9 phosphorylation and is identified and quantitated through measuring changes in non-phosphorylated β-catenin levels.

In a further embodiment activation of the GSK-3β is either through dephosphorylation or through phosphorylation of tyrosine 216 (Y216). GSK-3β phosphorylation can be identified and quantitated through changes in the molecular weight of the GSK-3β using methods described forthwith in the description. In one aspect of this embodiment the diagnostic kit of the present invention uses antibodies to identify and quantify GSK-3β expression levels.

It is a preferred embodiment of the present invention that GSK-3β is a diagnostic and/or therapeutic molecule incorporated into a diagnostic and/or therapeutic kit for detection and monitoring of GVHD.

It is a preferred embodiment of the diagnostic kit of the present invention to characterize GSK3β activity by detecting and quantifying the protein levels of total GSK3β and GSK3β phosphorylated at ser 9. Also in this embodiment, the diagnostic kit is able to detect and quantify GSK3β inactivation through activated protein kinase C (PKC) protein level and GSK3β inactivation through activated Wnt.

Thus it is a preferred embodiment of the present invention to identify GSK-3β associated molecular markers, for example proteins that are affected by the activated Wnt pathway or stimulated by activated PKC to increase the accuracy of GVHD detection. the site on the GSK-3β that is affected by the activated Wnt pathway and the site on the GSK-3β that is stimulated by activated PKC.

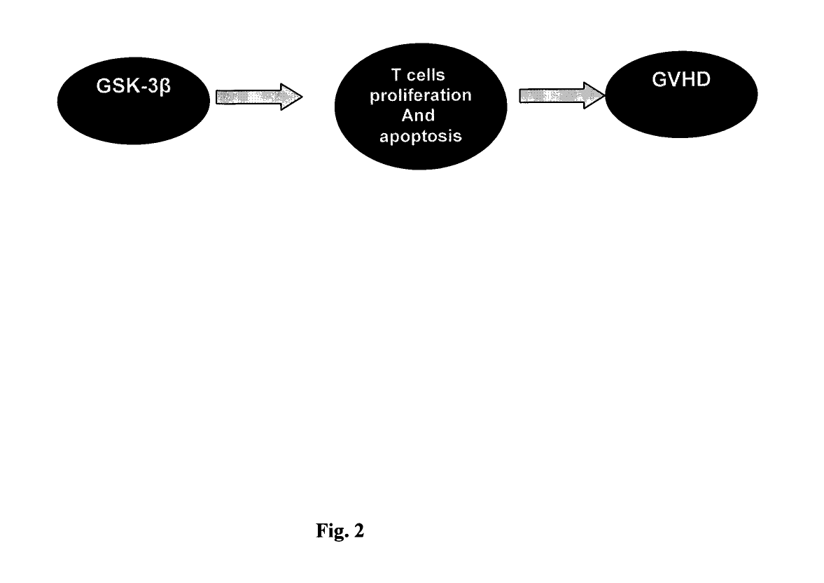

Abnormal cell proliferation and apoptosis is a main feature of Graft versus host disease (GVHD) and other conditions related to immune responses in which there is proliferation of T-cells as part of the patient's response to a foreign body or bodies as shown in FIG. 2 of the drawings. In this embodiment and other preferred embodiments the diagnostic molecule and kit is used and has applications in GVHD.

It is a further embodiment of the present invention that regulation of GSK-3β activation status by identification and quantification of the expression levels and inactivation and activation status of GSK-3β and its associated pathways is adapted to regulate and monitor GVHD. In this embodiment GSK-3β expression level is detected and quantified by the diagnostic kit of the present invention.

A diagnostic kit capable of identifying GVHD in at least phase 2 of the disease by identifying and quantifying the GSK-3β expression and activation status, is a further embodiment of the present invention. In this embodiment the diagnostic kit measures a range of parameters associated with GSK-3β activation states and associated pathways, and compares the deviation from standard measurements taken from healthy subjects in order to determine the extent of GVHD progress and make the appropriate diagnosis of GVHD.

The diagnostic kit of the present invention is adapted to detect the early onset of GVHD by examining changes in the GSK-3β activity by measuring or determining the expression levels of GSK-3β and related proteins Furthermore in this embodiment the diagnostic kit and methods of the present invention will be able to determine the cause of the GVHD with regard to T cell proliferation and apoptosis by determining how the GSK-3β regulates the aforementioned proliferation and apoptosis. In this aspect of the preferred embodiment the information provided by the diagnostic kit is used for determining the most effective treatment for the GVHD through regulating activation and/or inactivation status of the GSK-3β.

It is a further preferred embodiment of the present invention that the diagnostic kit is adapted to detect protein profiles specific and uniquely characteristic to GVHD so as to be able to make a correct and accurate diagnosis of GVHD. In this embodiment the unique protein profile prevents the diagnosis of false positive GVHD.

It is therefore a preferred embodiment of the present invention that GSK-3β acts as a regulatory and therapeutic molecule in a therapeutic or pharmaceutical kit in applications including but not limited to GVHD and tissue rejection.

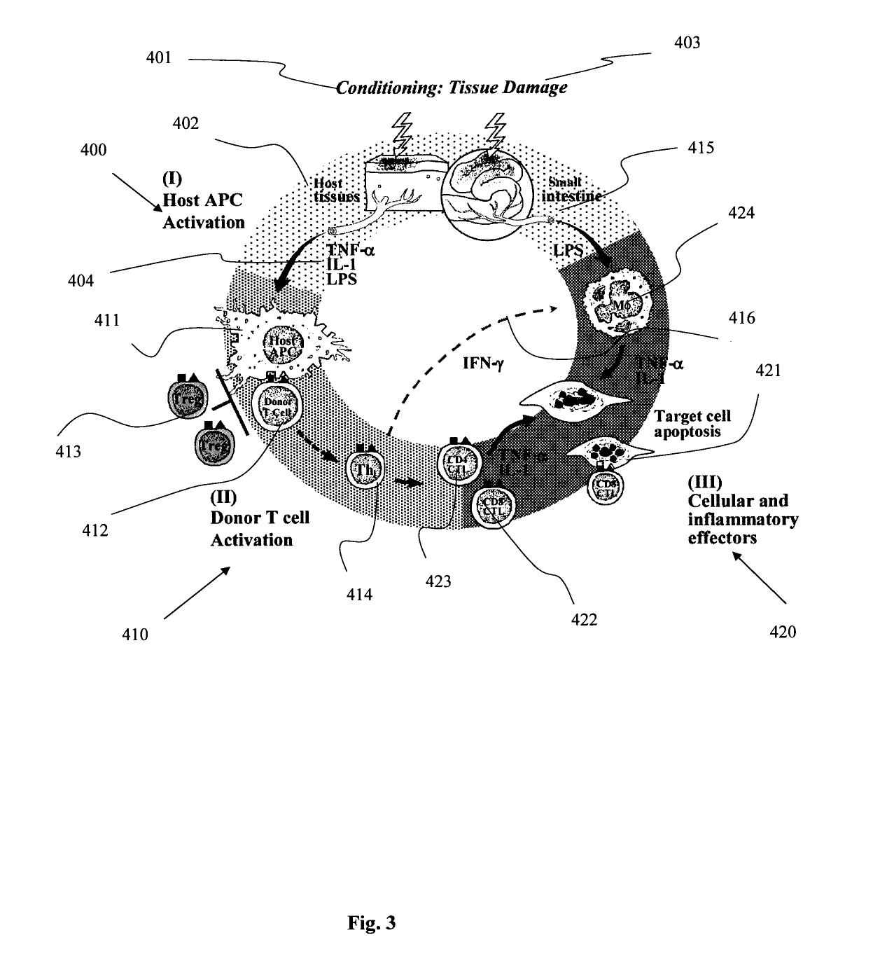

Reference is now made to FIG. 3 in the drawings in which 3 phases of GVHD disease progression are shown. In phase I 400, antigen presenting cells (APC) are activated, in the process of the recipient conditioning regimen 401, host tissue 402 is damaged 403, causing the release of inflammatory cytokines 404 such as Tumor Necrosis Factor α (TNF-α), Interleukin-1 (IL-1), Interleukin-6 (IL-6) and Lipopolysaccharide (LPS) immune complexes. Increased levels of these cytokines leads to activation of the host APC's. In phase II of GVHD 410 donor T-cell activation occurs, the host APC's 411 activate donor T-cells 412, this may be prevented by regulatory T cells (Treg) 413. The subsequent increased survival (i.e. decreased apoptosis), proliferation, migration 416 and differentiation of the activated T-cells in phase II of GVHD produces additional effectors that mediate further tissue damage including cytotoxic T Lymphocytes, Natural Killer (NK) cells, TNFα and IL-1. LPS that has leaked through the damaged intestinal mucosa 415 triggers additional TNFα production. TNFα can damage tissue directly by inducing necrosis and apoptosis in the skin and gastrointestinal tract through either TNF receptors or the Fas pathway. TNFα plays a direct role in intestinal GVHD damage which further amplifies damage in the skin, liver and lung in a “cytokine storm”. In addition type 1 T helper cells (Th1) 414 are also released in phase II to facilitate the immune response by releasing cytokines and helping in presenting antigens to the T-cells. In phase III of GVHD 420, T-cells target the host tissue for destruction with a range of cellular and inflammatory effectors. Target cells 421 undergo apoptosis and CD4 423, CD8 422 and CTL expressing T-cells are present in the immune response. Macrophages 424 are mediated by LPS and interferon-γ (IFN-γ) in phase 3 of the GVHD disease process. Once activated the macrophages initiate a cascade of inflammatory responses, including the release of mediators of tissue injury effecting in particular tissues and organs that undergo constant proliferation—such as the gastrointestinal tract and the skin.

It is an embodiment of the present invention that a diagnostic kit capable of detecting and quantitating the expression and activation status of the GSK-3β (GSK-3β activation levels and GSK-3β inactivation levels) is able to diagnose GVHD during phase II of the disease, at the stage when T-cell proliferation and migration occur. The novel diagnostic kit is able to diagnose GVHD at a relatively early stage before extensive tissue damage occurs, in a quick and relatively non-invasive manner.

In a further embodiment of the present invention, the diagnostic kit for GVHD is able to determine the status of GSK-3β as measured by examining GSK-3β expression levels. The diagnostic kit of the present invention is adapted to accurate and differential diagnosis of GVHD by further detection of unique RNA or protein profiles for GVHD, so as to prevent misdiagnosis with other conditions involving T-cell proliferation and activation.

In another embodiment of the invention, a protein chip utilizing an array of antibodies, biomarkers or antigens bound to a glass slide or equivalent substrate is used to determine the expression levels of different GVHD relevant proteins in a given sample.

In another embodiment of the invention, a diagnostic kit based on a protein chip is used to diagnose GVHD before the appearance of clinical symptoms. In certain embodiments the kit contains a custom-made protein chip comprising antibodies which are adapted to monitor the activation state and/or expression level of the GSK-3β, related pathways, apoptosis markers, proliferation markers, and other biological and biochemical molecules. Such molecule candidates can be predetermined and analysed by western blot, FACS, and other fingerprinting techniques.

In other preferred embodiments of the invention, the establishment of the GVHD diagnostic kit and its efficacy is tested by the following stages:
GVHD mouse model—samples from mice are utilized to determine the array of antibodies that is loaded on the protein chip. The aforementioned chip is then tested on mouse samples.
Clinical trials—used to determine the efficacy of the protein chip (diagnostic kit prototype) and to predict the development of GVHD in human transplant patients.

In order to understand the invention and to see how it may be implemented in practice, a plurality of preferred embodiments will now be described, by way of non-limiting example only, with reference to the following examples.

Reference is now made to experiments made in a mouse model to determine the involvement of GSK-3β and other proteins from related pathways in GVHD and their potential use as markers for GVHD diagnosis at an early disease stage.

### Goal:

To demonstrate that the levels of GSK-3β and proteins from related pathways can be used as markers for GVHD diagnosis at an early disease stage.

### GVHD Experimental Mouse Model:

Samples were collected from blood and spleen on days 0, 1, 3, 4, 5, & 6 post-transplant, from the following 3 groups (at least 3 mice per group):
1. Untreated: injected with of plain medium (no irradiation). Expected result: healthy mice.
2. Syngeneic: Balb/c mice injected with a splenocyte transplant from matched donor Balb/c mice after irradiation. Expected result: short-term effects of irradiation and full recovery, no GVHD.
3. Allogeneic: Balb/c mice injected with a splenocyte transplant from unmatched donor C57Bl/6 mice after irradiation. Expected result: short term effects of irradiation and subsequent GVHD development.

### Summary of Results:

The levels of p-GSK-3β (ser-9 phosphorylated GSK-3β and total GSK-3β are much lower in GVHD mice than in both syngeneic and untreated control mice.
The abovementioned effect is seen over time in day's 3-6 post transplant.

### Example 1

### GVHD Mouse Model

### Spleen Results

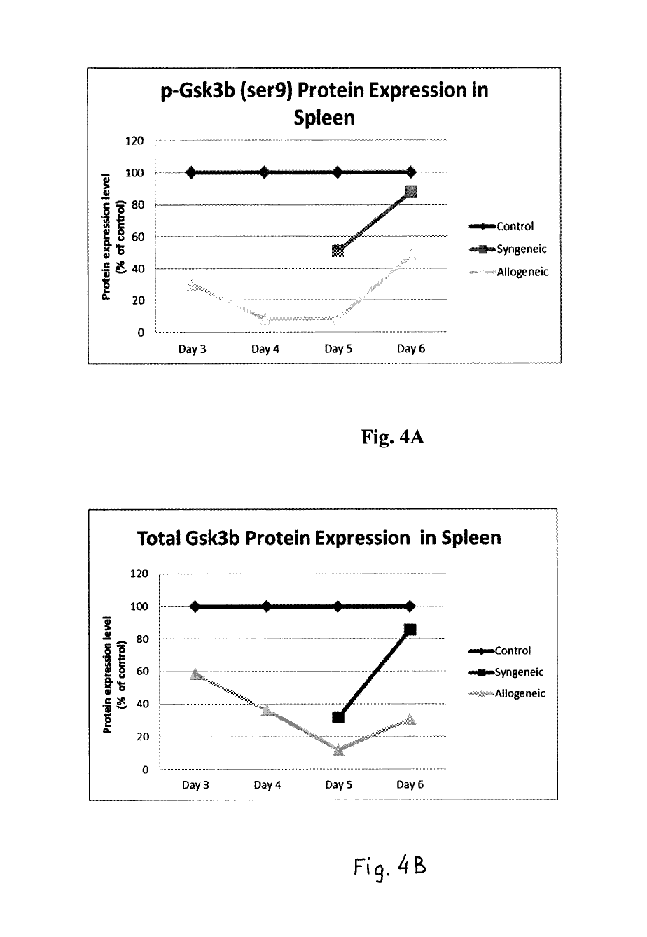

Reference is now made to FIG. 4 showing a graphical representation of the protein expression levels of p-GSK-3β (phosphotylated at ser 9 site) (FIG. 4A) and total GSK-3β (FIG. 4B) over time in spleen samples derived from Untreated (control), Syngeneic and Allogeneic mice.

The results have been normalized such that the values for the control mice were set as 100%. Each parameter was compared to the values of untreated mice.

As shown in FIG. 4, a prolonged decrease of total GSK-3β and p-GSK-3β over time was exclusively detected in the spleen of the allogeneic group mice.

It should be mentioned that in the Syngeneic group, the splenocyte population has not recovered enough to collect cells, until day 5.

In the Allogeneic group, proliferation of splenocytes was detected in the earlier days as a result of the immune response characteristic of GVHD.

On days 5 and 6, once severe symptoms have appeared in the mice, the levels of total and phosphorylated GSK-3β in the allogeneic mice were maintained at a lower level than the other groups, while in the syngeneic mice, the levels appear to approach almost normal.

Thus the results obtained show that, in the allogeneic group, p-GSK-3β and total GSK-3β decrease before the appearance of clinical symptoms (symptoms begin on day 4) and maintain lower than the untreated and Syngeneic groups throughout the disease course.

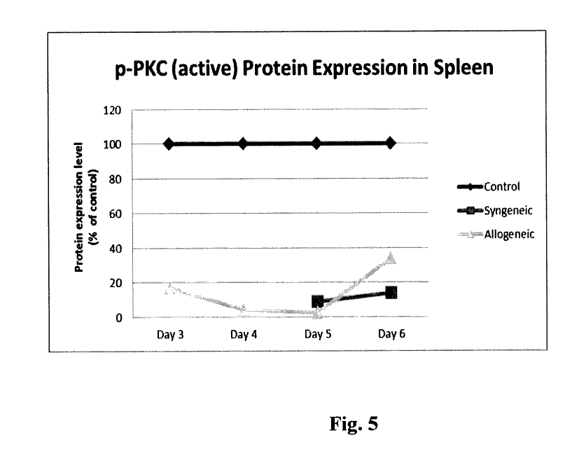

Reference is now made to FIG. 5 showing a graphical representation of the protein expression levels of p-PKC (active form), over time in spleen samples derived from Untreated (control), Syngeneic and Allogeneic mice.

It is herein acknowledged that PKC is located upstream of GSK-3β in several pathways, and is also capable of phosphorylating GSK-3β directly.

As shown in FIG. 5, the activated PKC levels are significantly lower in splenocytes of both Syngeneic and Allogeneic mice, relative to the control.

It should be emphasised that the results obtained have been normalized such that values of the control mice were set as 100%. Each parameter was compared to the value of untreated mice independently of other parameters.

### Conclusions for the Spleen Results:

The differences in the total GSK-3β and the p-GSK-3β in the spleen of the allogeneic mice compared to the syngeneic mice, can be detected as early as day 3, thus these proteins can be utilized as markers to identify and diagnose the early onset of GVHD in mice and potentially in humans.
Based on the above results, a diagnostic kit utilizing protein chip technology can be used to monitor the changes in the GSK-3β expression levels and changes in the activation state of GSK-3β in the spleen in order to predict and detect the early onset of GVHD in humans.

### Example 2

### GVHD Mouse Model

### Blood Results

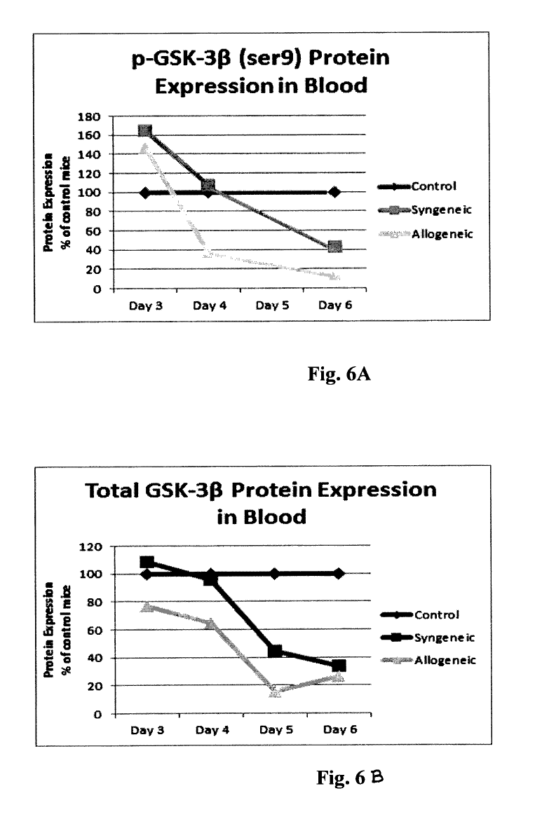

Reference is now made to FIG. 6 showing a graphical representation of the protein expression levels of p-GSK-3β (phosphotylated at ser 9 site) (FIG. 6A) and total GSK-3β (FIG. 6B) over time in blood samples derived from Untreated (control), Syngeneic and Allogeneic mice.

As shown in FIG. 6, a prolonged decrease of total GSK-3β and p-GSK-3β in the blood was observed over time only in the allogeneic group.

In the Syngeneic group, although a decrease in total GSK-3 and p-GSK-3β is observed, it appears in a later phase than in the allogeneic mice.

In the Allogeneic group, there is a decrease in the levels of total GSK-3β and p-GSK-3β over time, which is greater than the decrease observed in the syngeneic group.

It should be mentioned that the results have been normalized such that values for the control mice were set as 100%. Each parameter was compared to the values of the untreated mice.

### Conclusions for the Blood Results:

The decrease in total GSK-3β and p-GSK-3β in the blood detected in the allogeneic mice was greater than both the control and the syngeneic mice.
The decrease is most substantial on days 3 and 4, which are at the early stage of GVHD.
Based on the above results, a diagnostic kit utilizing protein chip technology can be used to monitor the changes in the GSK-3β expression levels and changes in the activation state of GSK-3β in the blood in order to predict and detect the early onset of GVHD in other mammals including humans.
Blood samples indicative of GVHD were obtained from mice, demonstrating the ability to collect usable blood samples from other mammals including humans.
Without wishing to be bound by theory, it is herein acknowledged that the detected blood levels in mice reflect that GVHD associated proliferation occurred in the spleen. Thus it can be assumed that the cells observed in the syngeneic mice on days 3, 4, and maybe 5 are cells that have existed in the periphery before the transplant. This is in contrast to the cells observed in the allogenenic mice on day 4, which are new cells that have been emerged from the spleen.

The above results demonstrate for the first time that GSK-3β is directly associated with the progression and onset of the GVHD disorder.

### Example 3

### Fingerprinting GVHD Disease

A further main aspect of the present invention is to explore the unique fingerprint of the GVHD disease. Such a fingerprint allows the establishment of a diagnostic kit as disclosed inter alia which comprises a set of proteins that are uniquely correlated with GVHD. In order to determine the fingerprint for GVHD, mouse model samples are analyzed by mass spectrometry to identify the following data:
The proteins that are present in the analysed system
Changes in protein expression
Changes in protein phosphorylation

Based on the analysis of the mouse samples, a custom-made protein chip is prepared. The kit includes candidate molecules identified to be involved in GVHD such as antibodies against GSK-3β, or proteins involved in related pathways, apoptosis markers, proliferation markers, and others (obtained by using techniques such as western blot analysis, FACS, and fingerprinting data). The aforementioned kit is used for prognosis of GVHD and its diagnosis in early stages.

Candidate antibodies used for the GVHD protein chip include antibodies recognizing the following biomarkers:
Total GSK-3β
GSK-3β ser 9 phosphorylation indicates GSK-3β inactivation
PKC Activation indication of GSK-3β inactivation
b-catenin—inactivation of the GSK-3β through the activated Wnt Pathway
Dvl—a protein upstream of GSK-3β in the Wnt pathway, and an indicator of Wnt pathway activation
Akt—PI-3K signaling cascade protein which phosphorylates GSK-3β at ser9.
Erk—Map kinase signaling cascade protein that directly phosphorylates GSK-3β at Ser9.
P38 MAPK—Map kinase signaling cascade protein that directly phosphorylates GSK-3β at Ser389.
GSK-3β Tyrosine 216 Phosphorylation—Indicates activation of GSK-3β
Serum Albumin—has been shown to be potentially linked to GVHD onset
Markers for apoptosis (Fas, Bcl family, Cyctochrome C, caspases)
Markers for immune activity (Nf-kB, CD25)
Markers for proliferation (Cyclin D1, PCNA, p27)

In further embodiments of the invention, the protein chip results are further validated by performing a comparison with western blot data to determine the efficacy, the sensitivity, and the accuracy of the chip.

The abovementioned information is used to assess the pathways which are regulating GSK-3β in GVHD and other pathways involved in this disease.

It is a main aspect of the invention that the changes seen in the GSK-3β expression levels and activation state are herein utilized as novel markers for diagnosing the GVHD. By monitoring the changes in the GSK-3β expression levels and changes in the activation state of the GSK-3β protein, the early onset of GVHD can be detected prior to observable clinical symptoms.

In another preferred embodiment of the invention, the use of GSK-3β as a marker allows the diagnosis of GVHD prior to the phase where observable symptoms are demonstrated by the patients and prior to the phase where the host body is attacked by the T-cells.

In further embodiments of the invention, Western blot analysis of mouse splenocyte lysates is performed using at least one of the following proteins as potential markers for GVHD:
b-catenin—a marker for the inactivation of the GSK-3β through the activated Wnt Pathway.
Dvl—a protein upstream of GSK-3β in the Wnt pathway, and an indicator of Wnt pathway activation.
Akt—PI-3K signaling cascade protein which phosphorylates GSK-3β at ser9.
Erk—Map kinase signaling cascade protein that directly phosphorylates GSK-3β at Ser9.
P38 MAPK—Map kinase signaling cascade protein that directly phosphorylates GSK-3β at Ser389.
GSK-3β tyr216 phosphorylation—Indicates the activation of the GSK-3β.

In further embodiments of the invention, Western blot analysis of mouse blood lysates is performed using GSK-3β, p-GSK-3β, PKC, and at least one of the antibodies listed above as potential markers for GVHD.

In a further embodiment of the invention, livers from GVHD model mice are used to perform analysis of GSK-3β and related proteins using western blot and/or mass spectrometry techniques or any other technique used for protein analysis.

It is also within the scope of the present invention that the regulation of GSK-3β expression is tested in a time-course study of an acute GVHD mouse model. Thus by GSK-3β manipulation GVHD onset can be prevented.

In a further embodiment of the invention, the substrates of the GSK-3β and the products associated with said substrates are identified.

In a further embodiment of the invention, the aforementioned substrates and products which are specific for the GVHD disorder are found. Those substrates and products are used for increasing the sensitivity of the diagnostic technology provided by the present invention.

In a further embodiment of the invention the specific pathway that stimulates the specific change in the activation state of the GSK-3β associated with the specific disorder of interest such as GVHD is identified. Such candidates that stimulate the activation state of the GSK-3β may include:
Dsh—Wnt pathway (upstream of GSK-3β)
Associated with inactivation of the GSK-3β through the activated Wnt pathway
Akt—PI-3K/Akt pathway
Associated with Ser 9 inactivation of the Wnt pathway
cAMP Associated with Ser 9 inactivation of the GSK-3β

The above described information indicates the pathways that are regulating the activation state of the GSK-3β in the specific disorder of interest such as GVHD. These results are used for fingerprinting a specific disease associated with activation state of the GSK-3β. Such fingerprinting data is used to increase the sensitivity of the diagnostic kit for the disease of interest.

### Example 4

### Clinical Study Using the GVHD Diagnostic Kit

The following clinical study is conducted in association with a CRA (clinical research associate), including Helsinki approval and negotiations with medical centers:
Patients for the clinical trial are recruited (including Helsinki approval and negotiations with medical centers).
Blood samples are collected from the patients who have undergone bone marrow transplants (throughout the year).
The protein chip is utilized for analysis of the human samples to test its efficacy in predicting acute GVHD onset. This is a retrospective study. The kit analysis results are compared against the diagnostic outcome (according to current medical standards) for each patient.
If necessary, the kit is calibrated by assaying human samples using western blot analysis.

1-52. (canceled)

53. A method of detecting GVHD in a mammal including a human comprising the steps of;
a. obtaining a sample from the patient or subject;
b. measuring the level of GSK-3b;
wherein a significant deviation from normal values indicates the presence of GVHD.

54. The method according to claim 53, further comprising at least one of the following steps: (a) measuring at least one parameter selected from a group consisting of GSK-3b expression levels, GSK-3b activation levels, GSK-3b inactivation levels, serine 9 phosphorylated GSK-3b expression and p-PKC expression. measuring at least one activation indicator selected from the group including over-expression of GSK-3b, tyrosine 216 phosphorylation or any combination thereof; (b) determining the expression and/or inactivation state of GSK-3b by measuring at least one inactivation indicator selected from the group consisting of serine 9 phosphorylation, Wnt activation as determined by b-catenin levels or through a protein marker specific for inactivation of the GSK-3β through the activated Wnt pathway or through the site on the GSK-3β that the activated Wnt pathway affects to stimulate inactivation of the GSK-3β; PKC expression levels or through a protein marker specific for inactivation of the GSK-3β through activated PKC, or any combination thereof; and (c) measuring expression and/or activation of GSK-3b by measuring at least one activation indicator selected from the group including over-expression of GSK-3b, tyrosine 216 phosphorylation or any combination thereof.

55. The method according to claim 53, comprising an additional step of sampling at least one of the spleen, the blood and a body fluid or tissue.

56. The method according to claim 53, comprising an additional step of comparing the result of said GSK-3b levels with a predetermined normal value obtained from healthy subjects or wherein a deviation of at least about 25% from normal inactivation values indicates the presence of GVHD.

57. The method according to claim 53, comprising additional steps of;
a. measuring the level of GSK-3b inactivation by at least one assay selected from the group consisting of measuring β-catenin levels in the absence of serine 9 phosphorylation, o measuring the site of stimulation on the GSK-3β once stimulated through the activated Wnt pathway, measuring a protein marker that is unique to the inactivation of GSK-3β through the activated Wnt pathway and measuring a protein marker that is unique to the inactivation of GSK-3β through the activated PKC; and
b. comparing the result of said inactivation levels with a predetermined normal inactivation value obtained from healthy subjects,
wherein a deviation of at least about 25% from normal inactivation values indicates the presence of GVHD.

58. The method according to claim 57, comprising an additional step of sampling at least one of the blood and the spleen.

59. A diagnostic kit for the identification and diagnosis of a pathological process associated with GVHD, wherein the diagnostic molecule is GSK-3b.

60. The kit according to claim 59, wherein the kit is adapted for detecting at least one parameter selected from a group consisting of: the expression level of GSK-3b, expression of p-PKC and the activation status of GSK-3b.

61. The kit according to claim 59, wherein said GVHD is detectable during phase 2 of the disease.

62. The kit according to claim 59, wherein the activation status and/or expression level of GSK-3b is a marker for the progression and/or presence of GVHD.

63. The kit according to claim 59, wherein at least one of the following holds true: (a) said kit is adapted for the detection and quantification of the activation and/or expression status of GSK-3b (b) said activation and/or expression status is an indicator of the cause of GVHD (c) said activation and/or expression status is an indicator for the best treatment program and (d) said pathological process is tissue rejection.

64. The diagnostic kit according to claim 62, wherein the activation status and/or expression level of GSK-3b is determined by measuring at least one parameter selected from a group consisting of: markers for the Wnt pathway or the actual site on the GSK-3β being affected by the activated Wnt pathway, serine 9 phosphorylation markers on the GSK-3β molecule, PKC expression or a protein marker specific for activated PKC inactivation of the GSK-3β, over-activation of GSK-3b, over-inactivated GSK-3b and any combination thereof.

65. The diagnostic kit according to claim 64, wherein over-activation of GSK-3b is determined by measuring tyrosine 216 phosphorylation or by measuring the expression levels of GSK-3β.

66. The diagnostic kit according to claim 62, provided with the means for detecting the presence of over-inactivated GSK-3b as indicated by at least one of the parameters selected from a group consisting of abnormal levels of β-catenin in the absence of serine 9 phosphorylation, abnormal levels of stimulation at the site on the GSK-3β affected by the activated Wnt pathway, abnormal levels of a protein marker that is unique to the inactivation of GSK-3β through the activated Wnt pathway, total GSK-3b, abnormal levels of stimulation at the site on the GSK-3β affected by activated PKC, abnormal levels of a protein marker that is unique to the inactivation of GSK-3β through activated PKC, abnormal levels of markers for Wnt activity, abnormal levels of β-catenin expression, abnormal levels of phosphorylated serine 9 GSK-3b, abnormal levels of markers for activated PKC inactivation of the GSK-3β and any combination thereof, thereby providing a positive diagnosis of GVHD.

67. The diagnostic kit according to claim 62, wherein the diagnostic kit characterizes the activation status or the expression status of GSK-3b so as to determine the cause of the GVHD.

68. The method according to claim 53, comprising additional steps of;
a. measuring GSK-3b inactivation indicators,
b. measuring GSK-3b activation indicators,
c. combining measurements of activation and inactivation indicators,
wherein an activation to inactivation ratio is obtained, characteristic of GVHD.

69. The diagnostic kit according to claim 59, wherein at least one of the following holds true: (a) the diagnostic kit uses further protein markers to further distinguish between GVHD and other pathological or normal physiological conditions that are also characterized by increased cell proliferation, decreased cell apoptosis and an immune response, (b) an additional positive detection of a unique RNA and/or protein profile for GVHD further verifies the presence of said GVHD during phase 2 of the disease, (c) the results provided by said kit indicate the pathways to be modulated to control the activation status and/or expression level of GSK-3b, thereby regulating GVHD.

70. A protein chip comprising an array of bound antibodies, biomarkers or antigens useful for determining the expression levels of different GVHD relevant proteins in a given sample.

71. The protein chip of claim 70, wherein at least one of the following holds true: (a) said kit comprising antibodies, biomarkers or antigens characteristic of the activation state and/or expression level of GSK-3β, related pathways, apoptosis markers, proliferation markers, and other biological and biochemical molecules relevant to GVHD wherein said antibodies, biomarkers or antigens are detectable before appearance of GVHD symptoms in a mammal including a human and (b) said antibodies, biomarkers or antigens are selected from the group comprising Total GSK-3β, ser 9 phosphorylated GSK-3β, GSK-3β ser 9 phosphorylation PKC Activation, b-catenin, Dvl, Akt, Erk, P38 MAPK, GSK-3β Tyrosine 216 Phosphorylation, Serum Albumin, markers for apoptosis comprising Fas, Bcl family, Cyctochrome C and caspases, markers for immune activity comprising Nf-kB and CD25, markers for proliferation comprising Cyclin D1, PCNA and p27), and any combination thereof.

72. A method of regulating the activation status and/or expression level of the GSK-3b in a in a mammal including a human comprising the steps of;
a. obtaining a blood sample from a patient or subject,
b. determining the activation status and/or expression level of GSK-3b,
c. regulating the activation status and/or expression level of GSK-3b according to the findings of the previous step, and
d. obtaining a normal activation status and/or expression level for GSK-3b
wherein regulation of said activation status and/or expression level of the GSK-3b is adapted for controlling GVHD.
